SEQUENCE LISTING (51 sequences)

SEQ ID NO: 1 - protein, 5 aa, Oryctolagus cuniculus
SYGVS

SEQ ID NO: 2 - protein, 7 aa, Oryctolagus cuniculus
IGSRGSA

SEQ ID NO: 3 - protein, 7 aa, Oryctolagus cuniculus
GFYDFDI

SEQ ID NO: 4 - protein, 7 aa, Oryctolagus cuniculus
IGSSGSA

SEQ ID NO: 5 - protein, 7 aa, Oryctolagus cuniculus
QSVYKNW

SEQ ID NO: 6 - protein, 4 aa, Oryctolagus cuniculus
KLAS

SEQ ID NO: 7 - protein, 13 aa, Oryctolagus cuniculus
LGSYGYSSGDCNA

SEQ ID NO: 8 - protein, 7 aa, Oryctolagus cuniculus
QNVYKNW

SEQ ID NO: 9 - protein, 7 aa, Oryctolagus cuniculus
QSVYSNW

SEQ ID NO: 10 - protein, 10 aa, Oryctolagus cuniculus
GFSLSSYGVS

SEQ ID NO: 11 - protein, 16 aa, Oryctolagus cuniculus
SIGSRGSAYCASWAKS

SEQ ID NO: 12 - protein, 9 aa, Oryctolagus cuniculus
ARGFYDFDI

SEQ ID NO: 13 - protein, 16 aa, Oryctolagus cuniculus
SIGSSGSAYYASWAKS

SEQ ID NO: 14 - protein, 16 aa, Oryctolagus cuniculus
GIGSRGSAYYASWAKS

SEQ ID NO: 15 - protein, 11 aa, Oryctolagus cuniculus
ASQSVYKNWLV

SEQ ID NO: 16 - protein, 7 aa, Oryctolagus cuniculus
DASKLAS

SEQ ID NO: 17 - protein, 11 aa, Oryctolagus cuniculus
SSQNVYKNWLA

SEQ ID NO: 18 - protein, 11 aa, Oryctolagus cuniculus
SSQSVYSNWLA

SEQ ID NO: 19 - protein, 7 aa, Artificial Sequence
  Synthetic Construct
  misc_feature (4)..(4): Xaa can be any naturally occurring amino acid
IGSXGSA

SEQ ID NO: 20 - protein, 15 aa, Artificial Sequence
  Synthetic Construct
  misc_feature (1)..(1): Xaa can be any naturally occurring amino acid
  misc_feature (5)..(5): Xaa can be any naturally occurring amino acid
  misc_feature (9)..(9): Xaa can be any naturally occurring amino acid
XIGSXGSAXASWAKS

SEQ ID NO: 21 - protein, 7 aa, Artificial Sequence
  Synthetic Construct
  misc_feature (2)..(2): Xaa can be any naturally occurring amino acid
  misc_feature (5)..(5): Xaa can be any naturally occurring amino acid
QXVYXNW

SEQ ID NO: 22 - protein, 11 aa, Artificial Sequence
  Synthetic Construct
  misc_feature (1)..(1): Xaa can be any naturally occurring amino acid
  misc_feature (4)..(4): Xaa can be any naturally occurring amino acid
  misc_feature (7)..(7): Xaa can be any naturally occurring amino acid
  misc_feature (11)..(11): Xaa can be any naturally occurring amino acid
XSQXVYXNWLX

SEQ ID NO: 23 - protein, 135 aa, Oryctolagus cuniculus
KGVQCQSVEESRGGLIKPTDTLTLTCTVSGFSLSSYGVSWVRQAPGTGLEWIGSIGSRGSAYCASWAKSRSTITRNTNLNTVTLKMTSLTAADTATYFCARGFYDFDIWGPGTLVTVSSGQPKAPSVFPLAPCCG

SEQ ID NO: 24 - protein, 135 aa, Oryctolagus cuniculus
KGVQCQSVEESRGGLIKPTDTLTLTCTVSGFSLSSYGVSWVRQAPGNGLEWIGSIGSSGSAYYASWAKSRSTITRNTNLNTVTLKMTSLTAADTATYFCARGFYDFDIWGPGTLVTVSSGQPKAPSVFPLAPCCG

SEQ ID NO: 25 - protein, 135 aa, Oryctolagus cuniculus
  misc_feature (6)..(6): Xaa can be any naturally occurring amino acid
KGVQCXSVEESRGGLIKPTDTLTLTCTVSGFSLSSYGVSWVRQAPGNGLEWIGGIGSRGSAYYASWAKSRSTITRNTNLNTVTLKMTSLTAADTATYFCARGFYDFDIWGPGTLVTVSSGQPKAPSVFPLAPCCG

SEQ ID NO: 26 - protein, 122 aa, Oryctolagus cuniculus
TFAQVLTQTPSSVSAAVGGTVTINCQASQSVYKNWLVWFQQKPGQRPKRLIYDASKLASGVSSRFKGSGSGTQFTLTISDLECDDAATYYCLGSYGYSSGDCNAFGGGTEVVVKSDPVAPTV

SEQ ID NO: 27 - protein, 122 aa, Oryctolagus cuniculus
TFAQVLTQTASPVSAAVGSTVTINCQSSQNVYKNWLAWFQQKPGQRPKRLIYDASKLASGVSSRFKGSGSGTQFTLTISDVQCDDAATYYCLGSYGYSSGDCNAFGGGTEVVVKGDPVAPTV

SEQ ID NO: 28 - protein, 122 aa, Oryctolagus cuniculus
TLAQVLTQTASPVSAAVGSTVTINCQSSQSVYSNWLAWFQQKPGQRPKRLIYDASKLASGVSSRFKGSGSGTQFTLTISGVQCDDAATYYCLGSYGYSSGDCNAFGGGTEVVVKGDPVAPTV

SEQ ID NO: 29 - DNA, 30 bp, Oryctolagus cuniculus
ggattctccctcagtagctatggggtgagt

SEQ ID NO: 30 - DNA, 30 bp, Oryctolagus cuniculus
ggattctccctcagtagctatggagtgagc

SEQ ID NO: 31 - DNA, 51 bp, Oryctolagus cuniculus
tccattggtagtcgtggtagcgcatactgcgcgagctgggcgaaaagccga

SEQ ID NO: 32 - DNA, 51 bp, Oryctolagus cuniculus
tccattggtagtagtggtagcgcatactacgcgagctgggcgaaaagccga

SEQ ID NO: 33 - DNA, 51 bp, Oryctolagus cuniculus
ggcattggtagccgtggtagtgcttactacgcgagctgggcgaaaagccga

SEQ ID NO: 34 - DNA, 27 bp, Oryctolagus cuniculus
gcgaggggattctacgactttgacatc

SEQ ID NO: 35 - DNA, 27 bp, Oryctolagus cuniculus
gcgagggggttctacgactttgacatc

SEQ ID NO: 36 - DNA, 27 bp, Oryctolagus cuniculus
gcgagggggttctacgactttgacatc

SEQ ID NO: 37 - DNA, 33 bp, Oryctolagus cuniculus
gccagtcagagtgtttataagaactggttagtt

SEQ ID NO: 38 - DNA, 33 bp, Oryctolagus cuniculus
tccagtcagaatgtttataagaactggttagcc

SEQ ID NO: 39 - DNA, 33 bp, Oryctolagus cuniculus
tccagtcagagtgtttatagtaactggttagcc

SEQ ID NO: 40 - DNA, 21 bp, Oryctolagus cuniculus
gatgcatccaaattggcatct

SEQ ID NO: 41 - DNA, 21 bp, Oryctolagus cuniculus
gatgcatcgaaactggcatct

SEQ ID NO: 42 - DNA, 39 bp, Oryctolagus cuniculus
ctaggcagttatggttatagtagtggtgattgtaatgct

SEQ ID NO: 43 - DNA, 39 bp, Oryctolagus cuniculus
ctaggcagttatgggtatagtagtggtgattgtaatgct

SEQ ID NO: 44 - DNA, 237 bp, Oryctolagus cuniculus
ggattctccctcagtagctatggggtgagttgggtccgccaggctccagggactgggctggaatggatcggatccattggtagtcgtggtagcgcatactgcgcgagctgggcgaaaagccgatccaccatcaccagaaacaccaacctgaacacggtaactctgaaaatgaccagtctgacagccgcggacacggccacctatttctgtgcgaggggattctacgactttgacatc

SEQ ID NO: 45 - DNA, 237 bp, Oryctolagus cuniculus
ggattctccctcagtagctatggagtgagctgggtccgccaggctccagggaacgggctggaatggatcggatccattggtagtagtggtagcgcatactacgcgagctgggcgaaaagccgatccaccatcaccagaaacaccaacctgaacacggtgactctgaaaatgaccagtctgacagccgcggacacggccacctatttctgtgcgagggggttctacgactttgacatc

SEQ ID NO: 46 - DNA, 237 bp, Oryctolagus cuniculus
ggattctccctcagtagctatggagtgagctgggtccgccaggctccagggaacgggctggaatggatcggaggcattggtagccgtggtagtgcttactacgcgagctgggcgaaaagccgatccaccatcaccagaaacaccaacctgaacacggtgactctgaaaatgaccagtctgacagccgcggacacggccacctatttctgtgcgagggggttctacgactttgacatc

SEQ ID NO: 47 - DNA, 234 bp, Oryctolagus cuniculus
gccagtcagagtgtttataagaactggttagtttggtttcagcagaaaccaggccagcgtcccaagcgcctgatctacgatgcatccaaattggcatctggggtctcatcgcggttcaaaggcagtggatctgggacacagttcactctcaccatcagcgacctggagtgtgacgatgctgccacttattactgtctaggcagttatggttatagtagtggtgattgtaatgct

SEQ ID NO: 48 - DNA, 234 bp, Oryctolagus cuniculus
tccagtcagaatgtttataagaactggttagcctggtttcagcagaaaccagggcagcgtcccaagcgcctaatctacgatgcatcgaaactggcatctggggtctcatcgcggttcaaaggcagtggatctgggacacaattcactctcaccatcagcgacgtgcagtgtgacgatgctgccacttactactgtctaggcagttatgggtatagtagtggtgattgtaatgct

SEQ ID NO: 49 - DNA, 234 bp, Oryctolagus cuniculus
tccagtcagagtgtttatagtaactggttagcctggtttcagcagaaaccagggcagcgtcccaagcgcctgatctacgatgcatcgaaactggcatctggggtctcatcgcggttcaaaggcagtggatctgggacacagttcactctcaccatcagcggcgtgcagtgtgacgatgctgccacttactactgtctgggcagttatgggtatagtagtggtgattgtaatgct

SEQ ID NO: 50 - protein, 9 aa, Homo sapiens
SEEKQNAVS

SEQ ID NO: 51 - DNA, 39 bp, Oryctolagus cuniculus
ctgggcagttatgggtatagtagtggtgattgtaatgct

(19) United States
(12) Patent Application Publication
(10) Pub. No.: US 2023/0203193 A1
(43) Pub. Date: Jun. 29, 2023

(54) ISOLATED ANTIBODIES AND FRAGMENTS THEREOF WITH SPECIFICITY FOR OSTEOPONTIN-C

(71) Applicant: GEORG F. WEBER, MILFORD, OH (US)

(72) Inventor: GEORG F. WEBER, MILFORD, OH (US)

(21) Appl. No.: 18/000,315

(22) PCT Filed: Jun. 2, 2021
(86) PCT No.: PCT/US2021/035379

§ 371 Date: Nov. 30, 2022

Related U.S. Application Data
(60) Provisional application No. 63/034,074, filed on Jun. 3, 2020.

(51) Int. Cl. C07K 16/32; G01N 33/574

(52) U.S. Cl. CPC C07K 16/32; G01N 33/57415; C07K 2317/565; C07K 2317/55

(57) ABSTRACT

This disclosure relates to isolated polypeptides with antigen specificity to OPN-c and to methods of their use for treating or determining or screening the presence of an invasive cancer. Included are three heavy chains CDRs and three light chain CDRs with binding specificity to OPN-c with little to no cross-reactivity to OPN-a and OPN-b. The ability to bind and detect OPN-c expression as well an OPN-c neutralization are also included.

### CROSS REFERENCE TO RELATED APPLICATIONS

This application claims priority to U.S. Provisional Patent Application 63/034,074, filed Jun. 3, 2020, the contents of which are hereby incorporated by reference in its entirety.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created Jun. 1, 2021, is named “OPNCmAb_SEQ_ST25.txt” and is 17,158 bytes in size.

### FIELD

This disclosure relates to isolated polypeptides with antigen specificity to OPN-c and to methods of their use for treating or determining or screening the presence of an invasive cancer.

### BACKGROUND

Osteopontin (OPN or bone sialoprotein I (BSP-1 or BNSP)) is a human protein encoded by the SPP1 gene on chromosome 4q13. Osteopontin was initially identified as an extracellular structural protein for bone formation and strength. However, its expression is not limited to bone tissues nor is its function limited to extracellular structure. Research has identified OPN as a negatively charged protein that interacts with many receptors and has ties to an array of signaling cascades, both extracellularly and intracellularly.

OPN proteins are encoded by the phosphoprotein 1 (SPP1) gene, which is housed on chromosome 4 (4q13). The SPP1 gene has 7 exons with multiple alternative splice variants identified. Importantly, three splice variants have been linked to cancer progression, OPN-a, OPN-b and OPN-c. OPN-b lacks exon 5 and OPN-c lacks exon 4. The variants of OPN have been linked to numerous cellular processes including anchorage-independence, migration, fusion and motility, all of which are significant abnormal malfunctions in malignant cancers.

Recently, an immunohistochemical study of exon 4 in samples from women with premalignant breast lesions identified that OPN-c expression is high in women who later progressed to have cancer [Walaszek et al., Br. J. Cancer 119: 1259-1266 2018]. In that study, low intensity staining revealed a correlation with survival five years after a sample was assessed, whereas high intensity correlated with a 30% chance of death within five years. The significance of these vast differences suggests OPN-c expression plays a significant role in breast cancer progression to malignancy. It is therefore desirable to interrupt the function or expression of OPN-c to slow tumor progression. Further, as OPN-c expression occurs early in those that progressed to cancer, detection of its expression enhances the chance to make an accurate prognosis. OPN-c has also been associated with the progression of other malignancies, including esophageal cancer, gastric cancer, glioma, liver cancer, lung cancer, mesothelioma, ovarian cancer, pancreatic cancer, prostate cancer and soft tissue sarcoma. Therefore, detection of OPN-c expression offers an early and reliable signal for the presence of malignant cells in a subject or a sample therefrom.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

In some aspects, the present disclosure concerns an isolated antibody to osteopontin-c (OPN-c), or an active fragment of the antibody. In certain aspects, the antibody is a monoclonal antibody. In some aspects, the antibody binds OPN-c but does not bind OPN-a or OPN-b. In further aspects, the antibody binds a polypeptide with an amino acid sequence as set forth in SEQ ID NO: 50.

In some aspects, the antibodies of the present disclosure include an isolated heavy chain (HC) variable domain polypeptide. In further aspects, the isolated HC variable domain polypeptide may include three HC complementarity determining regions (CDRs). In some aspects, the first HC CDR (HC CDR1) includes an amino acid sequence as set forth in SEQ ID NO: 1 or an active mutant thereof. In further aspects, the first HC CDR (HC CDR1) includes an amino acid sequence as set forth in SEQ ID NO: 10 or an active mutant thereof. In some aspects, the second HC CDR (HC CDR2) includes an amino acid sequence as set forth in IGSX₀GSA (SEQ ID NO: 19) or an active mutant thereof. In some aspects, X₀ is any amino acid. In further aspects, X₀ is an arginine or a serine. In other aspects, the second HC CDR (HC CDR2) includes an amino acid sequence as set forth in X₁IGSX₂GSAX₃ASWAKS (SEQ ID NO: 20) or an active mutant thereof. In some aspects, X₁ or X₂ or X₃ are any amino acid. In certain aspects, X₁ is glycine or serine, X₂ is arginine or serine, and X₃ is cysteine or tyrosine. In some aspects, the third HC CDR (HC CDR3) includes an amino acid sequence as set forth in SEQ ID NO: 3 or an active mutant thereof. In some aspects, the third HC CDR (HC CDR3) includes an amino acid sequence as set forth in SEQ ID NO: 12 or an active mutant thereof.

In further aspects, the present disclosure concerns OPN-c antibodies of isolated HC variable domains framework regions. In some aspects, the isolated HC variable domain includes an amino acid sequence with at least 85% identity to a sequence selected from SEQ ID NO: 23, SEQ ID NO: 24 or SEQ ID NO: 25. In some aspects, the isolated HC variable domain includes an amino acid sequence selected from SEQ ID NO: 23, SEQ ID NO: 24 or SEQ ID NO: 25.

In some aspects, the present disclosure concerns the antibodies that include an isolated light chain (LC) variable domain polypeptide. In some aspects, the isolated LC chain variable domain polypeptide includes three LC complementarity determining regions (CDRs). In some aspects, the first LC CDR (LC CDR1) includes an amino acid sequence as set forth in QX₄VYX₅NW (SEQ ID NO: 21) or an active mutant thereof. In some aspects, X₄ and X₅ are any amino acid. In certain aspects, X₄ is a serine or an asparagine. In further aspects, X₅ is a lysine or a serine. In some aspects, the first LC CDR (LC CDR1) includes an amino acid sequence as set forth in X₄SQX₅VYX₆NWLX₇ (SEQ ID NO: 22) or an active mutant thereof. In some aspects, X₄ or X₅ or X₆ or X₇ can be any amino acid. In certain aspects, X₄ is serine or asparagine and X₅ is lysine or serine and X₆ is alanine or serine, and X₇ is valine or alanine. In some aspects, the second LC CDR (LC CDR2) includes an amino acid sequence as set forth in SEQ ID NO: 6 or an active mutant thereof. In further aspects, the second LC CDR (LC CDR2) includes an amino acid sequence as set forth in SEQ ID NO: 16 or an active mutant thereof. In some aspects, the third LC CDR (LC CDR3) includes an amino acid sequence as set forth in SEQ ID NO: 7 or an active mutant thereof.

In some aspects, the present isolated LC variable domain polypeptides further include framework regions. In some aspects, the isolated LC variable domain includes an amino acid sequence with at least 85% identity to a sequence selected from SEQ ID NO: 26, SEQ ID NO: 27 or SEQ ID NO: 28. In other aspects, the isolated LC variable domain includes an amino acid sequence selected from SEQ ID NO: 26, SEQ ID NO: 27 or SEQ ID NO: 28.

In some aspects of the present disclosure, the antibody or active fragment thereof includes an Fv fragment. In certain aspects, the Fv fragment can be a single-chain Fv. In further aspects, the antibody or active fragment thereof may include a constant domain and a variable domain. In certain aspects, the antibody or active fragment thereof includes an Fab′ fragment.

In some aspects, the present disclosure concerns OPN-c antibodies or fragments thereof, where the antibody or active fragment thereof includes a human immunoglobulin framework domain grafted with the CDR sequences as set forth in SEQ ID NO: 1, SEQ ID NO: 19 and SEQ ID NO: 3. In some aspects, the antibody or active fragment thereof includes a human immunoglobulin framework domain grafted with the CDR sequences as set forth in SEQ ID NO: 10, SEQ ID NO: 20 and SEQ ID NO: 12. In some aspects, the antibody or active fragment thereof includes a human immunoglobulin framework domain grafted with the CDR sequences as set forth in SEQ ID NO: 21, SEQ ID NO: 6 and SEQ ID NO: 7. In further aspects, the antibody or active fragment thereof includes a human immunoglobulin framework domain grafted with the CDR sequences as set forth in SEQ ID NO: 22, SEQ ID NO: 16 and SEQ ID NO: 7.

In some aspects, the antibody or active fragment thereof is selected from immunoglobulin (Ig)A, IgD, IgE, IgG, or IgM.

In some aspects, the present disclosure concerns OPN-c antibodies or fragments thereof fused to additional proteins or peptides. In some aspects, the antibody or fragment can include a tag domain fused to a variable domain. In certain aspects, the tag domain may include a fluorescent protein or other fluorescent label, and/or a radiolabel. In certain aspects, the tag domain may include biotin or streptavidin. In other aspects, the antibody or fragment thereof can include a chemotherapeutic agent fused to a variable domain. In certain aspects, the chemotherapeutic agent may be an OPN-c inhibitor. An OPN-c inhibitor may include one or more of brefelamide, mesalazine, agelastatin A, trastuzumab, methotrexate or combinations thereof. In further aspects, the chemotherapeutic agent is a platinum containing agent.

In some aspects, the present disclosure also concerns polynucleotides encoding the isolated antibody or active fragments as set forth herein. In some aspects, the polynucleotide includes a sequence with at least 85% identity to the sequence set forth in SEQ ID NO: 29 or SEQ ID NO: 30 or as set forth in SEQ ID NO: 31 or SEQ ID NO: 32 or SEQ ID NO: 33 or as set forth in SEQ ID NO: 34 or SEQ ID NO: 35 or SEQ ID NO: 36 or as set forth in SEQ ID NO: 37 or SEQ ID NO: 38 or SEQ ID NO: 39 or as set forth in SEQ ID NO: 40 or SEQ ID NO: 41 or as set forth in SEQ ID NO: 44 or SEQ ID NO: 45 or SEQ ID NO: 46 or as set forth in SEQ ID NO: 47 or SEQ ID NO: 48 or SEQ ID NO: 49.

In some aspects, the present disclosure concerns methods of determining OPN-c expression in a tissue by obtaining a tissue sample from a subject and contacting the isolated antibody or active fragment thereof as described herein to the sample. Binding of the antibody or active fragment to the tissue sample may confirm expression of OPN-c in the subject's tissue.

In some aspects, the present disclosure concerns methods for determining risk of for malignancy from a pre-malignant breast lesion by obtaining a breast tissue sample from a subject with a pre-malignant lesion and contacting the isolated antibody or active fragment as described herein thereto. Binding of the anti-OPN-c antibody or active fragment thereof composition to the breast tissue sample signals an elevated risk for the subject. In some aspects binding of the isolated antibody or active fragment thereof is by immunohistochemical staining. In further aspects, the present disclosure concerns detecting OPN-c in a tissue or bodily fluid, such as blood, seminal fluid, milk, urine, bile, or saliva. In some aspects, the present disclosure concerns detecting OPN-c in blood or a fraction thereof, such as plasma or serum.

In some aspects, the present disclosure concerns a method of neutralizing OPN-c expression in a cell or the extracellular matrix thereof by administering the isolated antibody or active fragment thereof as set forth herein to a subject. Through the isolated antibody or active fragment thereof selectively binding to expressed OPN-c in the subject, the antibody or active fragment thereof can neutralize OPN-c activity within the cell or the extracellular matrix or the blood. In certain aspects, the isolated antibody or active fragment thereof binds an active domain within OPN-c to thereby neutralize function. In certain aspects, the isolated antibody or active fragment thereof includes a humanized antibody or fragment thereof. In further aspects, the isolated antibody or active fragment thereof includes a human immunoglobulin framework domain grafted with the CDR sequences as set forth in SEQ ID NO: 1, SEQ ID NO: 19 and SEQ ID NO: 3 or as set forth in SEQ ID NO: 10, SEQ ID NO: 20 and SEQ ID NO: 12 or as set forth in SEQ ID NO: 21, SEQ ID NO: 6 and SEQ ID NO: 7 or as set forth in SEQ ID NO: 22, SEQ ID NO: 16 and SEQ ID NO: 7. In further aspects, the isolated antibody or active fragment thereof is fused to a chemotherapeutic agent. In such aspects, the isolated antibody or active fragment thereof selectively binds expressed OPN-c to site specifically deliver the chemotherapeutic agent.

In some aspects, the present disclosure concerns an anti-osteopontin-c (OPN-c) binding composition that includes an isolated heavy chain (HC) variable domain polypeptide and an isolated light chain (HC) variable domain polypeptide. In some aspects, the HC variable domain includes three HC complementarity determining regions (CDRs), with the first HC CDR (HC CDR1) including an amino acid sequence as set forth in SEQ ID NO: 1, the second HC CDR (HC CDR2) including an amino acid sequence as set forth in SEQ ID NO: 19 and the third HC CDR (HC CDR3) including an amino acid sequence as set forth in SEQ ID NO: 3. In further aspects, the LC variable domain polypeptide includes three LC complementarity determining regions (CDRs), with the first LC CDR (LC CDR1) including an amino acid sequence as set forth in SEQ ID NO: 21, the second LC CDR (LC CDR2) including an amino acid sequence as forth in SEQ ID NO: 6, and the third LC CDR (LC CDR3) including an amino acid sequence as set forth in SEQ ID NO: 7. In certain aspects, the HC variable domain includes an amino acid sequence selected from SEQ ID NO: 23, SEQ ID NO: 24 or SEQ ID NO: 25 and the LC variable domain incudes an amino acid sequence selected from SEQ ID NO: 26, SEQ ID NO: 27 or SEQ ID NO: 28. In other aspects, the HC CDR2 includes an amino acid sequence as set forth in SEQ ID NO: 20. In other aspects still, the LC CDR1 includes an amino acid sequence as set forth in SEQ ID NO: 22.

In some aspects, the present disclosure concerns an isolated antibody or active fragment as set forth herein, with the antibody or active fragment thereof including an isolated heavy chain (HC) variable domain polypeptide and an isolated light chain (LC) variable domain. In some aspects, the HC variable domain includes three HC complementarity determining regions (CDRs), the first HC CDR (HC CDR1) including an amino acid sequence as set forth in SEQ ID NO: 1 or an active mutant thereof, the second HC CDR (HC CDR2) including an amino acid sequence as set forth in SEQ ID NO: 19 or an active mutant thereof and the third HC CDR (HC CDR3) including an amino acid sequence as set forth in SEQ ID NO: 3 or an active mutant thereof. In further aspects, the LC variable domain polypeptide includes three LC complementarity determining regions (CDRs), the first LC CDR (LC CDR1) including an amino acid sequence as set forth in SEQ ID NO: 21 or an active mutant thereof, the second LC CDR (LC CDR2) including an amino acid sequence as set forth in SEQ ID NO: 6 or an active mutant thereof, and the third LC CDR (LC CDR3) including an amino acid sequence as set forth in SEQ ID NO: 7 or an active mutant thereof. In some aspects, the antibody is a monoclonal antibody. In further aspects, the HC CDR2 includes an amino acid sequence as set forth in SEQ ID NO: 20. In even further aspects, the LC CDR1 includes an amino acid sequence as set forth in SEQ ID NO: 22.

### BRIEF DESCRIPTION OF THE DRAWINGS

The aspects set forth in the drawings are illustrative and exemplary in nature and not intended to limit the subject matter defined by the claims. The following detailed description of the illustrative aspects can be understood when read in conjunction with the following drawings.

FIG. 1A shows solid-phase ELISA for antibody-binding to GST-OPN splice variants OPN-a, OPN-b or OPN-c. OPNa B2, OPNa F1, and OPNa F2 are individual clones of the monoclonal antibodies to OPN-c. The antibody OPN MAB193P (BioRad) is an anti-panOPN antibody, and the antibody OPNa ahOPNc is a polyclonal chicken antibody to OPN splice variant-c. The y-axis indicates absorbance. The antibodies were tested for binding to OPN-a (top panel), OPN-b (middle panel), or OPN-c (bottom panel).

FIG. 1B shows the success of the antibodies utilized in FIG. 1A in further solid-phase ELISA binding to the distinct splice variants of GST-OPN.

FIG. 2 shows antibody neutralization of soft agar colony formation. Stably transfected MCF-7 OPN-c cells were plated in soft agar under standard conditions [He, B., Mirza, M. & Weber, GF. An osteopontin splice variant induces anchorage independence in human breast cancer cells. Oncogene 25: 2192-2202, 2006]. On day 0, 2 μg of antibody (clone B2) was added per plate and 0.6 μg every other day with medium (the control received only medium). The clone sizes were measured on day 11 as relative units [Shen H, Weber GF. The osteopontin-c splice junction is important for anchorage-independent growth. Mol. Carcinog. 53, 480-487, 2014]. The difference is significant according to the t-test (p=0.004).

### DESCRIPTION

Provided are isolated antibodies or fragments thereof with high specificity for OPN-c binding and compositions thereof, including lack of binding or recognition of OPN-a and OPN-b, and methods for their use. In some aspects, the compositions provided and described herein are capable of selectively binding SEQ ID NO: 50 (i.e., SEEKQNAVS). In some aspects, the present disclosure concerns antibodies that selectively bind OPN-c, such as monoclonal antibodies and/or polyclonal antibodies. Illustrative examples of compositions as provided herein include monoclonal antibodies (mAbs) or fragments thereof that are capable of selectively binding SEQ ID NO: 50. In some aspects, the present disclosure concerns compositions that do not bind or have a low affinity for OPN-a and/or OPN-b. In some aspects, the disclosure further relates to antibodies that do not recognize native OPN-a or OPN-b.

The terms “antibody” and “antibodies” as used herein include monoclonal antibodies, polyclonal, chimeric, single chain, bispecific, simianized, and humanized antibodies, as well as Fab fragments, including the products of a Fab immunoglobulin expression library. An intact antibody, a fragment thereof (e.g., Fab or F(ab′)₂), or an engineered variant thereof (e.g., sFv) can also be used. Optionally, an antibody is humanized as is recognized in the art. Optionally, an antibody is not humanized. Antibodies can be of any immunoglobulin class including IgG, IgM, IgE, IgA, IgD and any subclass thereof. In some aspects, the antibodies or fragments thereof of the present disclosure bind with high specificity to OPN-c. As used herein, the antibody or fragment thereof can be considered to be highly specific to OPN-c if the binding to OPN-c is of about 95% or higher and the binding to OPN-a and OPN-b is of about 5% or lower.

The term “CDR” as used herein refers to the “complementarity determining region” of an immunoglobulin (antibody) molecule. CDRs are part of the variable domain in an antibody where the antibody binds to its specific antigen. CDRs are crucial to the diversity of antigen specificities generated by lymphocytes. There are three CDRs per variable domain (i.e., CDR1, CDR2 and CDR3 in the variable domain of the light chain and CDR1, CDR2 and CDR3 in the variable domain of the heavy chain) for a total of 12 CDRs in an IgG molecule and 60 CDRs in an IgM molecule.

In some aspects, an antibody as provided herein is a humanized antibody. A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. Optionally, certain amino acids in the framework and constant domains of the heavy and/or light chains of the non-human species antibody may be mutated to produce the humanized antibody. Optionally, the constant domain(s) from a human antibody are fused to the variable domain(s) of a non-human species. Optionally, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

Antibodies or suitable fragments thereof as provided herein may be obtained from inoculation with an OPN-c specific peptide, such as SEQ ID NO: 50 (i.e. SEEKQNAVS). The isolated antibodies may be produced by initially inoculating an animal, such as a rabbit, mouse, chicken, pig, goat, rat or similar laboratory animal with a recombinant fragment of the OPN-c protein that observes the missing exon 4 or fragment thereof (i.e. aa 31-57 of SPP1) (including SEEKQNAVS (SEQ ID NO: 50)). After the isolation of plasma cells, cloning and expression, screening may be performed for monospecific cells to OPN-c, with further screening to confirm lack of recognition to OPN-a and OPN-b. The nucleotide sequences for the antibodies may then be identified by utilizing PCR (polymerase chain reaction) primers directed to the 5′ framework and 3′ constant regions that surround the complementarity determining regions (CDRs) or hyper variable domains (HVs)) of each chain.

Animals suitable for inoculation in the production of antibodies as provided herein are understood in the art, such as a vertebrate, including a mammalian or avian species. Antibodies may be generated in or obtained from a human, leporine, murine, bovine, porcine, ovine, camelidae, or equine species. Antibodies may be polyclonal (pAb) or monoclonal (mAb) in nature. Antibodies may further be processed, such as obtained humanized antibody versions that include CDRs obtained from a non-human species, such as CDRs from a rabbit monoclonal antibody (Rab mAb) in an otherwise human antibody sequence. As referenced above, polynucleotides encoding the antibodies may be readily obtained by techniques established in the art.

Antibodies as provided herein may include full-length antibodies or active fragments thereof that bind to or demonstrate affinity to SEQ ID NO: 50. Those skilled in the art will appreciate that recognition by the antibody toward the OPN-c antigen occurs through recognition by the variable domain of each chain of the antibody, with specificity coming from the complementarity determining regions (CDRs) therein. The CDRs are part of the variable domain in an antibody where the antibody binds to its specific antigen, such as OPN-c. For CDRs, there are typically three in each heavy chain and three in each light chain of the antibody (i.e., CDR1, CDR2 and CDR3 in the variable domain of the light chain and CDR1, CDR2 and CDR3 in the variable domain of the heavy chain). In some antibodies like immunoglobulin G (IgG) there are two light and two heavy chains, for a total of 12 CDRs. In an immunoglobulin M (IgM) there are 10 heavy chains and 10 light chains for a total of 60 CDRs in an IgM molecule. In some cases, however, recognition can occur through a single chain, such as through the 3 CDRs of a light chain (LC) or through the 3 CDRs of the heavy chain (HC). In fact, in some species (e.g. camelidae), a full length antibody can comprise a single heavy chain only. CDRs are crucial to the diversity of antigen specificities generated by lymphocytes. In some aspects of the present disclosure, there are three CDR per variable domain.

As such, this disclosure provides in some aspects anti-OPN-c antibody compositions, including full-length antibodies and active fragments thereof that optionally include one or more of the CDRs identified herein. The CDRs can include a variable domain when alternated within framework domains. Constant domains may also be used with the CDRs of this disclosure, including CL, CH₁, CH₂ and CH₃. The CDRs identified herein can be utilized within any full length antibody framework (or variable domain thereof), such as an IgG, IgM, IgD, IgA, IgE, IgY, or IgW. The CDRs may further be utilized within a human immunoglobulin framework.

This disclosure further concerns three identified monoclonal antibodies generated from a rabbit hybridoma model using the polypeptide SEQ ID NO: 50 as the initial inoculant or antigen. As presented in tables 1 and 2 and referred to throughout, the three monoclonal antibodies were identified and named as F2, F1 and B2. As set forth in their sequence alignments, each varied only in one CDR for each the heavy chain and the light chain: for all three, the heavy chain sequence only varied in CDR2 and the light chain only varied in CDR1. Additional silent mutations were observed in some polynucleotides encoding each.

TABLE 1
Minimum CDRs (or HVs) predicted for
Heavy and Light Chains

| mAb | | HEAVY CHAIN | LIGHT CHAIN |
|---|---|---|---|
| F2 | CDR 1 | SYGVS | QSVYKNW |
| | | (SEQ ID NO: 1) | (SEQ ID NO: 5) |
| | CDR 2 | IGSRGSA | KLAS |
| | | (SEQ ID NO: 2) | (SEQ ID NO: 6) |
| | CDR 3 | GFYDFDI | LGSYGYSSGDCNA |
| | | (SEQ ID NO: 3) | (SEQ ID NO: 7) |
| F1 | CDR 1 | SYGVS | QNVYKNW |
| | | (SEQ ID NO: 1) | (SEQ ID NO: 8) |
| | CDR 2 | IGSSGSA | KLAS |
| | | (SEQ ID NO: 4) | (SEQ ID NO: 6) |
| | CDR 3 | GFYDFDI | LGSYGYSSGDCNA |
| | | (SEQ ID NO: 3) | (SEQ ID NO: 7) |
| B2 | CDR 1 | SYGVS | QSVYSNW |
| | | (SEQ ID NO: 1) | (SEQ ID NO: 9) |
| | CDR 2 | IGSRGSA | KLAS |
| | | (SEQ ID NO: 2) | (SEQ ID NO: 6) |
| | CDR 3 | GFYDFDI | LGSYGYSSGDCNA |
| | | (SEQ ID NO: 3) | (SEQ ID NO: 7) |

Accepted algorithms contain slight differences in their prediction of the antibody binding domains. In some instances, the regions entail the sequences set forth in Table 2.

TABLE 2
Extended CDRs (or HVs) predicted for
Heavy and Light Chains

| mAb | | HEAVY CHAIN | LIGHT CHAIN |
|---|---|---|---|
| F2 | CDR 1 | GFSLSSYGVS | ASQSVYKNWLV |
| | | (SEQ ID NO: 10) | (SEQ ID NO: 15) |
| | CDR 2 | SIGSRGSAYCASWAKS | DASKLAS |
| | | (SEQ ID NO: 11) | (SEQ ID NO: 16) |
| | CDR 3 | ARGFYDFDI | LGSYGYSSGDCNA |
| | | (SEQ ID NO: 12) | (SEQ ID NO: 7) |
| F1 | CDR 1 | GFSLSSYGVS | SSQNVYKNWLA |
| | | (SEQ ID NO: 10) | (SEQ ID NO: 17) |
| | CDR 2 | SIGSSGSAYYASWAKS | DASKLAS |
| | | (SEQ ID NO: 13) | (SEQ ID NO: 16) |
| | CDR 3 | ARGFYDFDI | LGSYGYSSGDCNA |
| | | (SEQ ID NO: 12) | (SEQ ID NO: 7) |
| B2 | CDR 1 | GFSLSSYGVS | SSQSVYSNWLA |
| | | (SEQ ID NO: 10) | (SEQ ID NO: 18) |
| | CDR 2 | GIGSRGSAYYASWAKS | DASKLAS |
| | | (SEQ ID NO:14) | (SEQ ID NO: 16) |
| | CDR 3 | ARGFYDFDI | LGSYGYSSGDCNA |
| | | (SEQ ID NO: 12) | (SEQ ID NO: 7) |

It is understood in the art that the CDRs provide specific binding to the antigen (i.e. SEQ ID NO: 50), with the surrounding amino acid framework sequences being conserved, the framework providing structure to position the CDRs to bind antigen (e.g. a beta-sheet structure). Active mutants of the CDRs include those amino acid mutants or substitutions or additions to the sequences described herein that allow for the CDR mutant to retain antigen binding and/or recognition. Further conserved and further away from the variable domain are the constant regions, including the constant light chain domains (CL) and the constant heavy chain domains (CH). Those skilled in the art will recognize that characteristics and numbers of these can determine the class of full-length antibody (e.g. IgG etc.).

In some aspects, provided is an isolated heavy chain (HC) polypeptide including as CDR sequences SEQ ID NO: 1, SEQ ID NO: 3 and SEQ ID NO: 19 (IGSX₀GSA) or active mutants thereof. In some instances, the isolated heavy chain polypeptide includes SEQ ID NO: 1, SEQ ID NO: 3 and SEQ ID NO: 2 or SEQ ID NO: 1, SEQ ID NO: 3 and SEQ ID NO: 4 or active mutants thereof. In some instances for SEQ ID NO: 19, X₀ can be any amino acid, in other instances X₀ is arginine or serine.

Optionally, an isolated heavy chain (HC) polypeptide as provided herein includes as CDR sequences SEQ ID NO: 10, SEQ ID NO: 12 and SEQ ID NO: 20 (X₁IGSX₂GSAX₃ASWAKS) or active mutants thereof. In some instances, the isolated heavy chain polypeptide includes SEQ ID NO: 10, SEQ ID NO: 12 and SEQ ID NO: 11 or SEQ ID NO: 10, SEQ ID NO: 12 and SEQ ID NO: 13 or SEQ ID NO: 10, SEQ ID NO: 12 and SEQ ID NO: 14 or active mutants thereof. In some instances for SEQ ID NO: 20, X₁ or X₂ or X₃ can be any amino acid, in other instances X₁ is glycine or serine, X₂ is arginine or serine, and/or X₃ is cysteine or tyrosine.

Also provided herein are isolated light chain (LC) variable domain polypeptides that optionally include as CDR sequences SEQ ID NO: 6, SEQ ID NO: 7 and SEQ ID NO: 21 (QX₄VYX₅NW) or active mutants thereof. In some instances, the isolated light chain polypeptide includes SEQ ID NO: 5, SEQ ID NO: 6 and SEQ ID NO: 7 or SEQ ID NO: 8, SEQ ID NO: 6 and SEQ ID NO: 7 or SEQ ID NO: 9, SEQ ID NO: 6 and SEQ ID NO: 7 or active mutants thereof. In some instances for SEQ ID NO: 21, X₄ or X₅ can be any amino acid, in other instances X₄ is serine or asparagine and/or X₅ is lysine or serine.

Also provided are isolated light chain (LC) variable domain polypeptides that include as CDR sequences SEQ ID NO: 16, SEQ ID NO: 7 and SEQ ID NO: 22 (X₆SQX₄VYX₅NWLX₇) or active mutants thereof. In some instances, the isolated light chain polypeptide includes CDR sequences of SEQ ID NO: 15, SEQ ID NO: 16 and SEQ ID NO: 7 or SEQ ID NO: 17, SEQ ID NO: 16 and SEQ ID NO: 7 or SEQ ID NO: 18, SEQ ID NO: 16 and SEQ ID NO: 7 or active mutants thereof. In some instances for SEQ ID NO: 22, X₄ or X₅ or X₆ or X₇ can be any amino acid, in other instances X₄ is serine or asparagine and/or X₅ is lysine or serine and/or X₆ is alanine or serine, and/or X₇ is valine or alanine.

Each CDR may further include additional amino acids. SEQ ID NOs: 23-25 set forth exemplary variable domains comprised of the isolated heavy chain polypeptide CDR sequences and representative framework amino acids. SEQ ID NOs: 26-28 likewise represent isolated light chain polypeptides variable domains with the CDRs and framework amino acids. Each CDR may further include additional amino acids, such as one, two, three, four, five, six, seven, eight, nine, ten, eleven, twelve, thirteen, fourteen, or fifteen amino acids either toward the amino or the carboxyl terminal or both from the identified CDR sequences. CDR sequences can vary depending on the methodology utilized to identify such. For example, a Kabat derived sequence may differ slightly from an IMGT, or a Chothia or a Paratome identified sequence. (See, e.g., Wu et al., J. Exp. Med. 132: 211-250, 1970; Lefranc et al., Dev. Comp. Immunol. 27: 55-77, 2003; Chothia et al., J. Mol. Biol. 196: 901-917, 1987; and Kunik et al., PLoS Comput. Biol. 8: e1002388, 2012). As such, those skilled in the art will appreciate the potential for variance in the CDRs presented herein. The exemplary variable domains including framework amino acids are:

(F2 heavy chain):
(SEQ ID NO: 23)
KGVQCQSVEESRGGLIKPTDTLTLTCTVSGFSLSSYGVSWVRQAPGTGL
EWIGSIGSRGSAYCASWAKSRSTITRNTNLNTVTLKMTSLTAADTATYF
CARGFYDFDIWGPGTLVTVSSGQPKAPSVFPLAPCCG;
(F1 heavy chain):
SEQ ID NO: 24
KGVQCQSVEESRGGLIKPTDTLTLTCTVSGFSLSSYGVSWVRQAPGNGL
EWIGSIGSSGSAYYASWAKSRSTITRNTNLNTVTLKMTSLTAADTATYF
CARGFYDFDIWGPGTLVTVSSGQPKAPSVFPLAPCCG;
(B2 heavy chain):
SEQ ID NO: 25
KGVQCXSVEESRGGLIKPTDTLTLTCTVSGFSLSSYGVSWVRQAPGNGL
EWIGGIGSRGSAYYASWAKSRSTITRNTNLNTVTLKMTSLTAADTATYF
CARGFYDFDIWGPGTLVTVSSGQPKAPSVFPLAPCCG;
(F2 light chain):
SEQ ID NO: 26
TFAQVLTQTPSSVSAAVGGTVTINCQASQSVYKNWLVWFQQKPGQRPKR
LIYDASKLASGVSSRFKGSGSGTQFTLTISDLECDDAATYYCLGSYGYS
SGDCNAFGGGTEVVVKSDPVAPTV;
(F1 light chain):
SEQ ID NO: 27
TFAQVLTQTASPVSAAVGSTVTINCQSSQNVYKNWLAWFQQKPGQRPKR
LIYDASKLASGVSSRFKGSGSGTQFTLTISDVQCDDAATYYCLGSYGYS
SGDCNAFGGGTEVVVKGDPVAPTV;
(B2 light chain):
SEQ ID NO: 28
TLAQVLTQTASPVSAAVGSTVTINCQSSQSVYSNWLAWFQQKPGQRPKR
LIYDASKLASGVSSRFKGSGSGTQFTLTISGVQCDDAATYYCLGSYGYS
SGDCNAFGGGTEVVVKGDPVAPTV.

In some aspects, a composition as provided herein may be a combination of an isolated heavy chain and an isolated light chain. The two may be separate polypeptides or may be a fused, such as in a chimera. Those skilled in the art will appreciate that each may include a variable domain, comprised of CDRs (or HVs) and framework regions. The compositions may include a full length antibody or an active fragment thereof, such as a variable domain, an Fv domain, a scFv (single chain Fv), a Fab, an F(ab′)2, monospecific Fab2, bispecific Fab2, trispecific Fab3, monovalent immunoglobulin, bispecific diabody, trispecific triabody, scFv-Fc, and a minibody. Those skilled in the art will appreciate the different arrangements required for the structure of each.

Compositions of antibodies or active fragments thereof may include a single isolated heavy chain polypeptide or light chain polypeptide or may include multiple variants. For example, the variable domain of SEQ ID NO: 23 may be applied or expressed alone or in combination with SEQ ID NO: 24 and/or SEQ ID NO: 25, as can SEQ ID NO: 24 may be applied or expressed alone or in combination with SEQ ID NO: 23 and/or SEQ ID NO: 25 and SEQ ID NO: 25 may be applied or expressed alone or in combination with SEQ ID NO: 23 and/or SEQ ID NO: 24. Similarly for light chain polypeptides, they too may be used or expressed alone or in combination, such as SEQ ID NO: 26 as a single polypeptide or in concert with SEQ ID NO: 27 and/or SEQ ID NO: 28, or SEQ ID NO: 27 as a single polypeptide or in concert with SEQ ID NO: 26 and/or SEQ ID NO: 28 or SEQ ID NO: 28 as a single polypeptide or in concert with SEQ ID NO: 26 and/or SEQ ID NO: 27.

Those skilled in the art will further recognize that the isolated heavy chain polypeptides may be used or expressed with any isolated light chain polypeptide as described herein, as may the isolated light chain polypeptides be used or expressed with any isolated heavy chain polypeptide as described herein. For example, CDRs for F2 HC or an active mutant thereof can be paired with CDRs for F2 LC, F1 LC and/or B2 LC or active mutants thereof. CDRs for F1 HC or an active mutant thereof can be paired with F2 LC, F1 LC and/or B2 LC or active mutants thereof. CDRs for B2 HC or active mutants thereof can be paired with F2 LC, F1 LC and B2 LC or active mutants thereof.

It is further contemplated that in addition to the isolated amino acids sequences set forth herein, variants are also considered herein. For example, the CDRs or the variable domains set forth herein may have at least 80% sequence identity, for example at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-28.

The CDRs described herein may further be incorporated into a human antibody to provide a humanized anti-OPN-c antibody. A humanized antibody contains an amino acid sequence that differs from the sequence of the rabbit monoclonal antibodies disclosed herein by one or more amino acid substitutions, additions and/or deletions. The purpose of such changes being to minimize the likelihood that the humanized antibody will induce a human immune response or reduce the severity of a human immune response. Amino acids in the framework and constant domains of the heavy and/or light chains of the rabbit monoclonal (Rab mAb) amino acid sequences disclosed herein may be mutated to produce the humanized antibody with possibly the additional fusion of constant domain(s) from a human antibody to the humanized variable domain(s). It is a further possibility to change one or more amino acid residues in one or more CDR sequences disclosed herein to generate or increase or modify a specific human immune response. Such may occur when the changed amino acid residue(s) either are not critical for antigen recognition or at least where the changes are to a conservative amino acid substitution such that antigen binding and/or recognition is not dramatically impacted in a negative manner. Such may be typically accomplished through polynucleotide cloning techniques familiar in the art. For example, restriction enzymes and PCR can be utilized to excise and/or mutate certain bases for ligation into a polynucleotide encoding a human immunoglobulin. For example, a humanization strategy includes grafting the rabbit monoclonal (Rab mAb) CDRs sequences described herein onto the combined Kabat/IMTG/Paratome CDR regions of a human sequence to generate a stable humanized mAb producing germline. (See, e.g., Zhang et al., mAbs 9: 419-429, 2017 and Yu et al., PLoS One 5:e9072, 2010).

The CDR polypeptides, antibodies or active fragments thereof described herein may further be fused or linked with additional polypeptides and/or labels. Those skilled in the art will appreciate that further fused polypeptides may include expressed tags for purification purposes, such as a myc tag, a FLAG tag, a hexa-His (or poly-His) tag, an AP tag, a T7 tag, a V5 tag, a BCCP tag, a B-tag, a CBP tag, an E2 tag, a DHFR tag, an EE tag, a GST tag, an HA tag, an HRP tag, an HSV tag, a KT3 tag, a LacZ tag, a MBP tag, a poly-Arg tag, a poly-Asp tag, a poly-Cys tag, a poly-Phe tag, a Protein C tag, a S1 tag, a S tag, a streptavidin tag, a biotin tag, a Protein A tag, a Protein G tag, a Strep-tag, a SUMO tag, a TAP tag, a TrpE tag, and a VSV-G tag. Additional polypeptides may also include fluorescent proteins, such as GFP, BFP, mKalama, Sirius, Sapphire, CFP, cerulean, mTurquoise, Midoriishi-cyan, Azami green, mUKG, mWasabi, Clover, mNeonGreen, YFP, citrine, Venus, Kusabira-orange, mKOk, mOrange, mRaspberry, mCherry, mStrawberry, mTangerine, tdTomato, RFP, mApple, mRuby, mPlum, HcRed-tandem, mKate2, mNeptune, iRFP, mKeima Red, mBeRFP, Kaede, KikGR1, mEos2, mEOS 3 PSmOrange and Dronpa. In further aspects, the labels may include a fluorescent dye, such as a fluorescent dye coupled to an antibody as described herein or to a secondary antibody. Such dyes may include cyanine dyes (such as Cy3, Cy3.5, Cy5, Cy5.5, Cy7, Cy7.5), acridine orange, BCECF, aminomethylcoumarin, fluorescein, FITC, TRITC, R-phycoerythrin, Texas Red, Alexa Fluor dyes, Cascade Blue dyes, coumarin dyes, and DyLight dyes. Further contemplated are the inclusion of radiolabels, such as through the incorporation of radioisotopes, including ³H, ³²P, ¹⁴C, ¹²³I, ¹²⁵I, ¹³¹I, ⁶⁰Co, ¹³⁷Ce, and ⁵¹Cr.

For the antibodies and the active fragments thereof described herein, the heavy chain polypeptides and the light chain polypeptides can be expressed from an isolated polynucleotide encoding the amino acid sequences set forth herein. For example, a CDR1 for a heavy chain polypeptide can be encoded by the polynucleotide sequences as follow: ggattctccctcagtagctatggggtgagt (SEQ ID NO: 29) or ggattctccctcagtagctatggagtgagc (SEQ ID NO: 30). Similarly, a CDR2 for a heavy chain polypeptide can be encoded by the polynucleotide sequences as follow: tccattggtagtcgtggtagcgcatactgcgcgagctgggcgaaaagccga (SEQ ID NO: 31) or tccattggtagtagtggtagcgcatactacgcgagctgggcgaaaagccga (SEQ ID NO: 32) or ggcattggta gccgtggtag tgcttactacgcgagctgggcgaaaagccga (SEQ ID NO: 33). A CDR3 for a heavy chain polypeptide can be encoded by the polynucleotide sequences as follow: gcgaggggattctacgactttgacatc (SEQ ID NO: 34) or gcgagggggttctacgactttgacatc (SEQ ID NO: 35) or gcgagggggttctacgactttgacatc (SEQ ID NO: 36).

Further, a CDR1 for a light chain polypeptide can be encoded by the polynucleotide sequences as follow: gccagtcagagtgtttataagaactggttagtt (SEQ ID NO: 37) or tccagtcagaatgtttataagaactggttagcc (SEQ ID NO: 38) or tccagtcagagtgtttatagtaactggttagcc (SEQ ID NO: 39). A CDR2 for a light chain polypeptide can be encoded by the polynucleotide sequences as follow: gatgcatccaaattggcatct (SEQ ID NO: 40) or gatgcatcgaaactggcatct (SEQ ID NO: 41). A CDR3 for a light chain polypeptide can be encoded by the polynucleotide sequences as follow: ctaggcagttatggttatagtagtggtgattgtaatgct (SEQ ID NO: 42) or ctaggcagttatgggtatagtagtggtgattgtaatgct (SEQ ID NO: 43) or ctgggcagttatgggtatagtagtggtgattgtaatgct (SEQ ID NO: 51). Polynucleotides encoding the heavy chain CDRs with framework amino acids are set forth in SEQ ID NOs: 44-46 and the light chain CDRs with framework amino acids are set forth in SEQ ID Nos: 47-49 (SEQ ID NO: 44 ggattctccctcagtagctatggggtgagttgggtccgccaggctccagggactgggctggaatggatcggatccattggtagtcgtggta gcgcatactgcgcgagctgggcgaaaagccgatccaccatcaccagaaacaccaacctgaacacggtaactctgaaaatgaccagtctg acagccgcggacacggccacctatttctgtgcgaggggattctacgactttgacatc; SEQ ID NO: 45 ggattctccctcagtagctatggagtgagctgggtccgccaggctccagggaacgggctggaatggatcggatccattggtagtagtggta gcgcatactacgcgagctgggcgaaaagccgatccaccatcaccagaaacaccaacctgaacacggtgactctgaaaatgaccagtctg acagccgcggacacggccacctatttctgtgcgagggggttctacgactttgacatc; SEQ ID NO: 46 ggattctccctcagtagctatggagtgagctgggtccgccaggctccagggaacgggctggaatggatcggaggcattggtagccgtggt agtgcttactacgcgagctgggcgaaaagccgatccaccatcaccagaaacaccaacctgaacacggtgactctgaaaatgaccagtctg acagccgcggacacggccacctatttctgtgcgagggggttctacgactttgacatc; SEQ ID NO: 47 gccagtcagagtgtttataagaactggttagtttggtttcagcagaaaccaggccagcgtcccaagcgcctgatctacgatgcatccaaattg gcatctggggtctcatcgcggttcaaaggcagtggatctgggacacagttcactctcaccatcagcgacctggagtgtgacgatgctgcca cttattactgtctaggcagttatggttatagtagtggtgattgtaatgct; SEQ ID NO: 48 tccagtcagaatgtttataagaactggttagcctggtttcagcagaaaccagggcagcgtcccaagcgcctaatctacgatgcatcgaaact ggcatctggggtctcatcgcggttcaaaggcagtggatctgggacacaattcactctcaccatcagcgacgtgcagtgtgacgatgctgcc acttactactgtctaggcagttatgggtatagtagtggtgattgtaatgct; SEQ ID NO: 49 tccagtcagagtgtttatagtaactggttagcctggtttcagcagaaaccagggcagcgtcccaagcgcctgatctacgatgcatcgaaact ggcatctggggtctcatcgcggttcaaaggcagtggatctgggacacagttcactctcaccatcagcggcgtgcagtg tgacgatgctgccacttactactgtctgggcagttatgggtatagtagtggtgattgtaatgct).

It should further be noted that in some aspects of the present disclosure, additional polynucleotides may be included, such as further silent mutations in a polynucleotide sequence encoding a CDR. Such mutations or substitutions should be understood in the art, including: TTT and TTC to encode for phenylalanine; TTA, TTG, CTT, CTC, CTG to encode for leucine; TCT, TCC, TCA, TCG, AGT and AGC to encode for serine; TAT and TAC to encode for tyrosine; TGT and TGC to encode for cysteine; ATT, ATC and ATA to encode for isoleucine; AGA and AGG to encode for arginine; GAT and GAC to encode for aspartic acid; GGT, GGC, GGA and GGG to encode for glycine; GCT, GCC, GCA and GCG to encode for alanine; GTT, GTC, GTA and GTG to encode for valine; and, AAA and AAG to encode for lysine.

In addition to the polynucleotides sequences set forth herein, in some aspects of the present disclosure additional variants are also considered. For example, the CDRs or the variable domains as set forth herein may have at least 80% sequence identity, for example at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one or more of SEQ ID NOs: 29-49.

Those skilled in the art will appreciate that once obtained, multiple systems can be utilized for producing the antibodies described herein from polynucleotides encoding the same, such as through known cloning techniques including the use of: transfection, infection, transformation, viral vectors, plasmids, transgenic systems (e.g. CRE/lox), CRISPR/Cas systems, site-directed mutagenesis, co-expressed resistance/selection genes, ligations, restrictions and so on. Suitable expression systems are also understood in the art, including plant cells, bacterial cells (e.g. E. coli), yeast expression systems, mammalian cells lines, tumor derived cell lines, primary cell cultures, viral expression systems (e.g. modified/attenuated adenovirus) etc. Further details for particular cloning/expression techniques can be found, for example, at M. R. Green and J. Sambrook, Molecular Cloning: A Laboratory Manual, Cold Spring Harbor Laboratory Press; 4th Ed., 2012; F. M. Ausubel, Ed., Short Protocols in Molecular Biology, Current Protocols; 5th Ed., 2002; B. Alberts et al., Molecular Biology of the Cell, 4th Ed., Garland, 2002; CRISPR/Cas: A Laboratory Manual, Doudna and Mali (eds), Cold Spring Harbor Laboratory Press, Cold Spring Harbor, N.Y., USA, 2016.

Methods of Using Anti-OPN-c Antibodies or Active Fragments/Mutants Thereof

Further provided are methods of utilizing the antibodies or active fragments or mutants described herein. Many uses for application of the antibodies, antibody fragments, and compositions thereof will be readily apparent to those skilled in the art, and the following are for illustrative purposes rather than a demonstration of limitation.

In some aspects of the present disclosure, the methods can utilize the specific activity of the antibodies and/or fragments thereof toward binding OPN-c, as well as the lack of recognition to OPN-a and OPN-b. For example, binding of the antibodies and/or fragments thereof in a sample from a subject can confirm expression of OPN-c in the subject. A “subject” may refer to a mammal, such as a human or non-human primate, or a canine, feline, equine, ovine, bovine, leporine, porcine or murine species. A “tissue sample” or “sample” refers to material obtained from a biological organism, a tissue, cell, extracellular material or any medium suitable for mimicking biological conditions, or from the cellular micro-environment. In some instances, a tissue sample may include breast tissue. In some aspects, a tissue sample may include a bodily fluid, such as urine, saliva, milk, seminal fluid, blood, and bile. In certain aspects, as tissue sample may include a fractioned portion of a bodily fluid, such as serum or plasma from blood.

Confirmation of binding can be achieved through known techniques in the art. For example, labeling of the antibodies or using an intermediary antibody can help to visualize the presence of OPN-c, such as through immunohistochemistry methods, blotting, flow cytometry, radiodetection, tag detection and the like.

In some aspects, the antibodies and/or the active fragments thereof can further be used in methods for diagnosis, such as application as a diagnostic tool to assess for breast cancer or breast malignancies or abnormal cellular growth. As identified herein, expression of OPN-c can correlate to a poor prognosis or chance of survival in subjects with abnormal or malignant growth of cells associated with breast tissue. OPN-c is also associated with the progression of other malignancies, including, but not limited to, esophageal, gastric, liver, lung, ovarian, pancreatic and prostate cancers, as well as glioma, mesothelioma and soft tissue sarcoma. The methods can include adding the antibodies, antibody fragments or compositions including the same to a tissue sample and then confirming whether the monoclonal antibody or active fragment bound, such as through visualization means. In some aspects, a tissue sample may be obtained from a subject through techniques known in the art, such as with a biopsy or venipuncture. In some aspects, is further contemplated that the monoclonal antibodies or active fragments thereof can be administered for in vivo imaging. For example, in some aspects it may be of particular interest to identify the early expression of OPN-c in lesions, and as such performing these methods on samples from subjects presented with such may help to provide an earlier prognosis to increase aggressiveness of therapeutic intervention.

In some aspects, the antibodies and/or the active fragments thereof can further be used in methods for in vitro diagnosis. As set forth herein, contacting a tissue sample, such as a sample of breast tissue, a blood sample, a serum sample, a plasma sample, a milk sample, a urine sample, a saliva sample, a sample of seminal fluid, and/or a bile sample, with the antibodies and/or the active fragments thereof allows for determining the presence of OPN-c therein by confirming binding through techniques understood in the art, including immunohistochemistry and ELISA (enzyme-linked immunoabsorbent assay).

In some aspects, OPN-c expression in a tissue or tissue sample can be determined by ELISA, wherein the addition of an enzyme and substrate provides a detectable output, such as a color change to confirm the presence of OPN-c. In some instances, an enzyme or substrate may be coupled to the antibodies or fragments thereof as set forth herein. In other aspects, an ezyme or a substrate may be affixed to a panOPN-antibody. By affixing the enzyme or substrate thereof to an antibody that will bind OPN-c, the enzyme-substrate reaction is specific to the presence of OPN-c. Various approaches to ELISA may be utilized, such as direct ELISA, sandwich ELISA, competitive ELISA and reverse ELISA.

In some aspects, OPN-c expression in a tissue or tissue sample can be determined by immunohistochemistry. In such aspects, a tissue sample is further fixed to retain cell morphology and the architecture of the tissue. The fixed sample may then be sliced and incubated with antibodies or fragments thereof as described herein. In some aspects, the sliced, fixed sample may be blocked with a buffer to diminish non-specific binding. The antibody or fragments thereof to OPN-c can be incubated with the sliced, fixed sample and the binding can be confirmed wither through direct conjugation of a reporter to the antibody or fragment thereof or by further incubation with a secondary antibody or fragment thereof that is conjugated to a reporter. Reporters may include fluorescent proteins, fluorescent dyes, enzymes or substrates thereof. In some aspects, the sliced, fixed sample may be additionally counterstained to provide a contrast.

As also demonstrated herein, the OPN-c monoclonal antibodies demonstrate improved neutralization for some undesired effects that are correlated with OPN-c expression. One such effect is anchorage-independent survival. As demonstrated in the examples herein, the contact by the monoclonal anti-OPN-c antibodies disrupts adherent colony formation in cells expressing OPN-c. As such, the administration of or contacting with the monoclonal antibodies or active fragments thereof, either in vitro, in situ or in vivo may provide methods that allow for the disruption of cell survival and expansion in tumor or lesion cells. It will also be appreciated by those skilled in the art that application or administration of humanized antibodies or active fragments thereof comprised of the CDRs disclosed herein can neutralize OPN-c with no or minimal adverse reaction to the antibody itself, lending such to be of further benefit in instances of human in vivo application or administration.

Further, as the antibodies and antibody fragments as set forth herein demonstrate selectivity for OPN-c, coupling the antibodies or antibody fragments to a chemotherapeutic agent offers method for achieving advanced site specific delivery. Due to the high selectivity of the antibodies and/or active fragments thereof, toxic chemotherapeutic compounds such as platinum containing agents, may be better employed to target specific cells expressing OPN-c, as well as OPN-c itself, thereby allowing for site-specific delivery and/or reduced doses of toxic chemotherapeutic compounds. Further, coupling to the monoclonal antibodies or the active fragments thereof with other pan-OPN inhibitory agents offers a further way to selectively target OPN-c activity. While agents such as brefelamide, mesalazine, agelastatin A, trastuzumab, and methotrexate may not selectively target OPN or OPN related pathways, coupling to the monoclonal antibodies offers an advanced site specific delivery to target cells expressing OPN-c over OPN-a and OPN-b.

Various aspects of the present disclosure are illustrated by the following non-limiting examples. The examples are for illustrative purposes and are not a limitation on any practice of the present invention. It will be understood that variations and modifications can be made without departing from the spirit and scope of the invention.

### EXAMPLES

A peptide reflecting the continuous sequence in OPN-c (i.e. missing amino acids 31-57, SEQ ID NO: 50) was prepared and utilized as an inoculant in rabbits. Plasma cells were obtained and cell lines were generated, which were screened for antibodies to bind the inoculant. Three IgG antibodies were obtained and further evaluated, i.e. F2, F1 and B2.

The nucleotide sequences encoding the CDRs were obtained by utilizing PCR with 5′ and 3′ primers to conserved domains, followed by routine nucleotide sequencing. The identity and variance within the CDRs was then observed by predictive translation of the sequences.

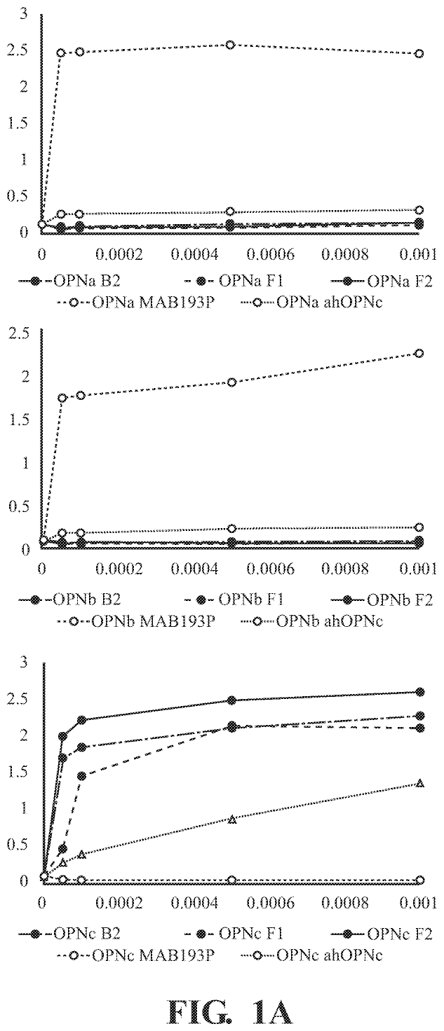

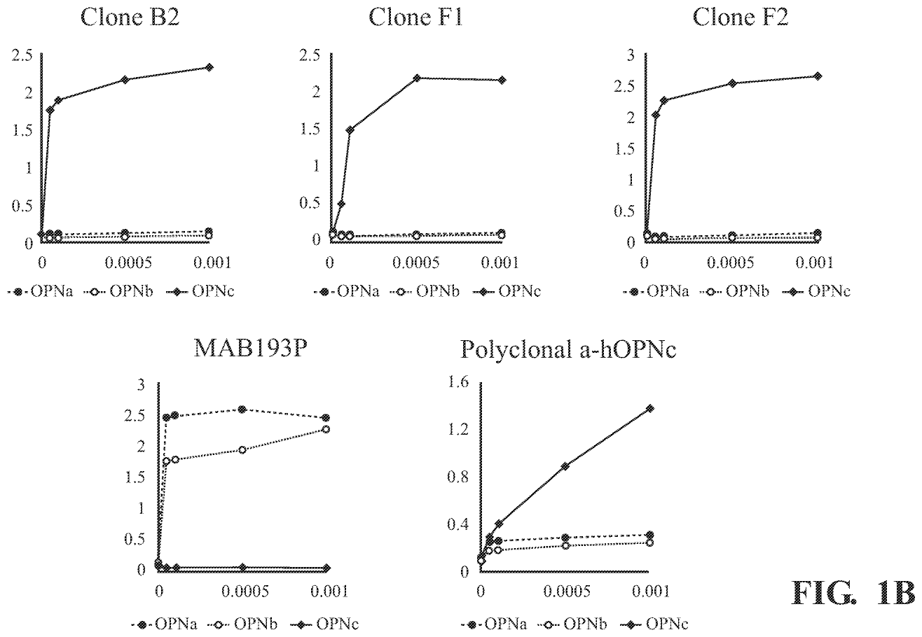

The binding ability of each obtained monoclonal antibody was then tested. FIGS. 1A and 1B show ELISA data concerning the specific binding to OPN-c. The B2, F1, and F2 individual clones of the monoclonal antibodies were compared to an anti-panOPN antibody and to a polyclonal chicken antibody to OPN splice variant-c. FIG. 1A shows the various antibodies binding to OPN-a (top panel), OPN-b (middle panel), or OPN-c (bottom panel). The data show that the three monoclonal antibodies had high selectivity for OPN-c and outperformed the previous polyclonal Ab. FIG. 1B shows individual antibodies binding to the distinct splice variants of GST-OPN. Again the three monoclonal antibodies show great selectivity for OPN-c with negligible cross reactivity to either OPN-a or OPN-b.

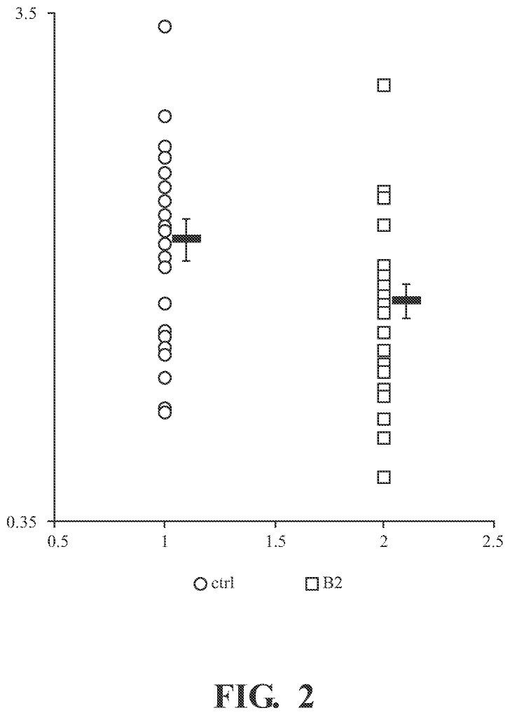

Next, antibody neutralization of soft agar colony formation was examined. Stably transfected MCF-7 OPN-c cells were plated in soft agar under standard conditions [He et al. Oncogene 25, 2192-2202, 2006]. On day 0, 2 μg of antibody (clone B2) was added per plate followed by 0.6 μg every other day with medium (the control received only medium). The clone sizes were measured on day 11 as relative units [as per Shen et al. Mol. Carcinog. 53:480-487, 2014]. The obtained data show that the monoclonal antibodies effectuated a disruption to the ability of OPN-c expressing cells to effectively form colonies (see FIG. 2). As OPN-c is important for tumor cell deadherent survival, these data reveal that the monoclonal antibodies, in addition to selectively binding OPN-c can further neutralize some innate activity.

### Further Examples

Example 1. An isolated antibody to osteopontin-c (OPN-c) or an active fragment thereof, wherein the isolated antibody or active fragment thereof comprises an isolated heavy chain (HC) variable domain polypeptide or comprises an isolated light chain (HC) variable domain polypeptide or comprises an isolated HC variable domain and an isolated LC variable domain.

Example 2. An isolated antibody or active fragment thereof of Example 1, wherein the isolated antibody or active fragment thereof binds OPN-c but does not bind OPN-a or OPN-b.

Example 3. The isolated antibody or active fragment thereof of Example 1 or 2, wherein the isolated antibody or active fragment thereof binds a polypeptide with an amino acid sequence as set forth in SEQ ID NO: 50.

Example 4. An isolated antibody to osteopontin-c (OPN-c) or an active fragment thereof, wherein the isolated antibody or active fragment thereof comprises an isolated heavy chain (HC) variable domain polypeptide.

Example 5. The isolated antibody or active fragment thereof of any of Examples 1-4, wherein the isolated HC variable domain polypeptide comprises three HC complementarity determining regions (CDRs).

Example 6. The isolated antibody or active fragment thereof of any of Examples 1-5, wherein the first HC CDR (HC CDR1) is comprised of an amino acid sequence as set forth in SEQ ID NO: 1 or an active mutant thereof.

Example 7. The isolated antibody or active fragment thereof of any of Examples 1-5, wherein the first HC CDR (HC CDR1) is comprised of an amino acid sequence as set forth in SEQ ID NO: 10 or an active mutant thereof.

Example 8. The isolated antibody or active fragment thereof of any of Examples 1-5, wherein the second HC CDR (HC CDR2) is comprised of an amino acid sequence as set forth in IGSX₀GSA (SEQ ID NO: 19) or an active mutant thereof.

Example 9. The isolated antibody or active fragment thereof of Example 8, wherein X₀ is any amino acid.

Example 10. The isolated antibody or active fragment thereof of Example 8, wherein X₀ is an arginine or a serine.

Example 11. The isolated antibody or active fragment thereof of any of Examples 1-5, wherein the second HC CDR (HC CDR2) is comprised of an amino acid sequence as set forth in X₁IGSX₂GSAX₃ASWAKS (SEQ ID NO: 20) or an active mutant thereof.

Example 12. The isolated antibody or active fragment thereof of Example 11, wherein X₁ or X₂ or X₃ are any amino acid.

Example 13. The isolated antibody or active fragment thereof of Example 11, wherein X₁ is glycine or serine, X₂ is arginine or serine, and X₃ is cysteine or tyrosine.

Example 14. The isolated antibody or active fragment thereof of any of Examples 1-5, wherein the third HC CDR (HC CDR3) is comprised of an amino acid sequence as set forth in SEQ ID NO: 3 or an active mutant thereof.

Example 15. The isolated antibody or active fragment thereof of any of Examples 1-5, wherein the third HC CDR (HC CDR3) is comprised of an amino acid sequence as set forth in SEQ ID NO: 12 or an active mutant thereof.

Example 16. The isolated antibody or active fragment thereof of any of Examples 1-4, wherein the isolated HC variable domain polypeptide further comprises framework regions.

Example 17. The isolated antibody or active fragment thereof of any of Examples 1-16, wherein the isolated HC variable domain comprises an amino acid sequence with at least 85% identity to a sequence selected from the group consisting of SEQ ID NO: 23, SEQ ID NO: 24 or SEQ ID NO: 25.

Example 18. The isolated antibody or active fragment thereof of Example 17, wherein the isolated HC variable domain comprises an amino acid sequence selected from the group consisting of SEQ ID NO: 23, SEQ ID NO: 24 or SEQ ID NO: 25.

Example 19. An isolated antibody to osteopontin-c (OPN-c) or an active fragment thereof, wherein the isolated antibody or active fragment thereof comprises an isolated light chain (LC) variable domain polypeptide.

Example 20. The isolated antibody or active fragment thereof of any of Examples 1-19, wherein the isolated LC chain variable domain polypeptide comprises three LC complementarity determining regions (CDRs).

Example 21. The isolated antibody or active fragment thereof of any of Examples 1-20, wherein the first LC CDR (LC CDR1) is comprised of an amino acid sequence as set forth in QX₄VYX₅NW (SEQ ID NO: 21) or an active mutant thereof.

Example 22. The isolated antibody or active fragment thereof of Example 21, wherein X₄ and X₅ are any amino acid.

Example 23. The isolated antibody or active fragment thereof of Example 21, wherein X₄ is a serine or an asparagine.

Example 24. The isolated antibody or active fragment thereof of Example 21, wherein X₅ is a lysine or a serine.

Example 25. The isolated antibody or active fragment thereof of Example 21, wherein the first LC CDR (LC CDR1) is comprised of an amino acid sequence as set forth in X₄SQX₅VYX₆NWLX₇ (SEQ ID NO: 22) or an active mutant thereof.

Example 26. The isolated antibody or active fragment thereof of Example 25, wherein X₄ or X₅ or X₆ or X₇ can be any amino acid.

Example 27. The isolated antibody or active fragment thereof of Example 25, wherein X₄ is serine or asparagine and X₅ is lysine or serine and X₆ is alanine or serine, and X₇ is valine or alanine.

Example 28. The isolated antibody or active fragment thereof of any of Examples 1-20, wherein the second LC CDR (LC CDR2) is comprised of an amino acid sequence as set forth in SEQ ID NO: 6 or an active mutant thereof.

Example 29. The isolated antibody or active fragment thereof of any of Examples 1-20, wherein the second LC CDR (LC CDR2) is comprised of an amino acid sequence as set forth in SEQ ID NO: 16 or an active mutant thereof.

Example 30. The isolated antibody or active fragment thereof of any of Examples 1-20, wherein the third LC CDR (LC CDR3) is comprised of an amino acid sequence as set forth in SEQ ID NO: 7 or an active mutant thereof.

Example 31. The isolated antibody or active fragment thereof of any of Examples 1-20, wherein the isolated LC variable domain polypeptide further comprises framework regions.

Example 32. The isolated antibody or active fragment thereof of any of Examples 1-31, wherein the isolated LC variable domain comprises an amino acid sequence with at least 85% identity to a sequence selected from the group consisting of SEQ ID NO: 26, SEQ ID NO: 27 or SEQ ID NO: 28.

Example 33. The isolated antibody or active fragment thereof of any of Examples 1-20, wherein the isolated LC variable domain comprises an amino acid sequence selected from the group consisting of SEQ ID NO: 26, SEQ ID NO: 27 or SEQ ID NO: 28.

Example 34. An isolated antibody to osteopontin-c (OPN-c) or an active fragment thereof, wherein the isolated antibody or active fragment thereof comprises an isolated HC variable domain and an isolated LC variable domain polypeptide.

Example 35. The isolated antibody or active fragment thereof of any of Examples 1-34, wherein the isolated HC variable domain polypeptide comprises three HC complementarity determining regions (CDRs).

Example 36. The isolated antibody or active fragment thereof of any of Examples 1-35, wherein the first HC CDR (HC CDR1) is comprised of an amino acid sequence as set forth in SEQ ID NO: 1 or an active mutant thereof.

Example 37. The isolated antibody or active fragment thereof of any of Examples 1-35, wherein the first HC CDR (HC CDR1) is comprised of an amino acid sequence as set forth in SEQ ID NO: 10 or an active mutant thereof.

Example 38. The isolated antibody or active fragment thereof of any of Examples 1-35, wherein the second HC CDR (HC CDR2) is comprised of an amino acid sequence as set forth in IGSX₀GSA (SEQ ID NO: 19) or an active mutant thereof.

Example 39. The isolated antibody or active fragment thereof of Example 38, wherein X₀ is any amino acid.

Example 40. The isolated antibody or active fragment thereof of Example 38, wherein X₀ is an arginine or a serine.

Example 41. The isolated antibody or active fragment thereof of any of Examples 1-35, wherein the second HC CDR (HC CDR2) is comprised of an amino acid sequence as set forth in X₁IGSX₂GSAX₃ASWAKS (SEQ ID NO: 20) or an active mutant thereof.

Example 42. The isolated antibody or active fragment thereof of Example 41, wherein X₁ or X₂ or X₃ are any amino acid.

Example 43. The isolated antibody or active fragment thereof of Example 41, wherein X₁ is glycine or serine, X₂ is arginine or serine, and X₃ is cysteine or tyrosine.

Example 44. The isolated antibody or active fragment thereof of any of Examples 1-35, wherein the third HC CDR (HC CDR3) is comprised of an amino acid sequence as set forth in SEQ ID NO: 3 or an active mutant thereof.

Example 45. The isolated antibody or active fragment thereof of any of Examples 1-35, wherein the third HC CDR (HC CDR3) is comprised of an amino acid sequence as set forth in SEQ ID NO: 12 or an active mutant thereof.

Example 46. The isolated antibody or active fragment thereof of any of Examples 1-34, wherein the isolated HC variable domain polypeptide further comprises framework regions.

Example 47. The isolated antibody or active fragment thereof of any of Examples 1-46, wherein the isolated HC variable domain comprises an amino acid sequence with at least 85% identity to a sequence selected from the group consisting of SEQ ID NO: 23, SEQ ID NO: 24 or SEQ ID NO: 25.

Example 48. The isolated antibody or active fragment thereof of any of Examples 1-47, wherein the isolated HC variable domain comprises an amino acid sequence selected from the group consisting of SEQ ID NO: 23, SEQ ID NO: 24 or SEQ ID NO: 25.

Example 49. The isolated antibody or active fragment thereof of any of Examples 1-34, wherein the isolated LC chain variable domain polypeptide comprises three LC complementarity determining regions (CDRs).

Example 50. The isolated antibody or active fragment thereof of any of Examples 1-49, wherein the first LC CDR (LC CDR1) is comprised of an amino acid sequence as set forth in QX₄VYX₅NW (SEQ ID NO: 21) or an active mutant thereof.

Example 51. The isolated antibody or active fragment thereof of Example 50, wherein X₄ and X₅ are any amino acid.

Example 52. The isolated antibody or active fragment thereof of Example 50, wherein X₄ is a serine or an asparagine.

Example 53. The isolated antibody or active fragment thereof of Example 50, wherein X₅ is a lysine or a serine.

Example 54. The isolated antibody or active fragment thereof of any of Examples 1-50, wherein the first LC CDR (LC CDR1) is comprised of an amino acid sequence as set forth in X₄SQX₅VYX₆NWLX₇ (SEQ ID NO: 22) or an active mutant thereof.

Example 55. The isolated antibody or active fragment thereof of Example 54, wherein X₄ or X₅ or X₆ or X₇ can be any amino acid.

Example 56. The isolated antibody or active fragment thereof of Example 54, wherein X₄ is serine or asparagine and X₅ is lysine or serine and X₆ is alanine or serine, and X₇ is valine or alanine.

Example 57. The isolated antibody or active fragment thereof of any of Examples 1-49, wherein the second LC CDR (LC CDR2) is comprised of an amino acid sequence as set forth in SEQ ID NO: 6 or an active mutant thereof.

Example 58. The isolated antibody or active fragment thereof of any of Examples 1-49, wherein the second LC CDR (LC CDR2) is comprised of an amino acid sequence as set forth in SEQ ID NO: 16 or an active mutant thereof.

Example 59. The isolated antibody or active fragment thereof of any of Examples 1-49, wherein the third LC CDR (LC CDR3) is comprised of an amino acid sequence as set forth in SEQ ID NO: 7 or an active mutant thereof.

Example 60. The isolated antibody or active fragment thereof of any of Examples 1-34, wherein the isolated LC variable domain polypeptide further comprises framework regions.

Example 61. The isolated antibody or active fragment thereof of any of Examples 1-60, wherein the isolated LC variable domain comprises an amino acid sequence with at least 85% identity to a sequence selected from the group consisting of SEQ ID NO: 26, SEQ ID NO: 27 or SEQ ID NO: 28.

Example 62. The isolated antibody or active fragment thereof of any of Examples 1-34, wherein the isolated LC variable domain comprises an amino acid sequence selected from the group consisting of SEQ ID NO: 26, SEQ ID NO: 27 or SEQ ID NO: 28.

Example 63. The isolated antibody or active fragment of any of Examples 1-62, wherein the isolated antibody or active fragment thereof is a monoclonal antibody.

Example 64. The isolated antibody or active fragment thereof of any of Examples 1-62, wherein the isolated antibody or active fragment thereof is an Fv fragment.

Example 65. The isolated antibody or active fragment thereof of Example 64, wherein the Fv is a single-chain Fv.

Example 66. The isolated antibody or active fragment thereof of any of Examples 1-62, wherein the isolated antibody or active fragment thereof further comprises a constant domain.

Example 67. The isolated antibody or active fragment thereof of Example 66, wherein the isolated antibody or active fragment thereof is an Fab′ fragment.

Example 68. The isolated antibody or active fragment thereof of any of Examples 1-62, wherein the isolated antibody or active fragment thereof further comprises a human immunoglobulin framework domain grafted with the CDR sequences as set forth in SEQ ID NO: 1, SEQ ID NO: 19 and SEQ ID NO: 3.

Example 69. The isolated antibody or active fragment thereof of any of Examples 1-62, wherein the isolated antibody or active fragment thereof further comprises a human immunoglobulin framework domain grafted with the CDR sequences as set forth in SEQ ID NO: 10, SEQ ID NO: 20 and SEQ ID NO: 12.

Example 70. The isolated antibody or active fragment thereof of any of Examples 1-62, wherein the isolated antibody or active fragment thereof further comprises a human immunoglobulin framework domain grafted with the CDR sequences as set forth in SEQ ID NO: 21, SEQ ID NO: 6 and SEQ ID NO: 7.

Example 71. The isolated antibody or active fragment thereof of any of Examples 1-62, wherein the isolated antibody or active fragment thereof further comprises a human immunoglobulin framework domain grafted with the CDR sequences as set forth in SEQ ID NO: 22, SEQ ID NO: 16 and SEQ ID NO: 7.

Example 72. The isolated antibody or active fragment thereof of any of Examples 1-62, wherein the isolated antibody or active fragment thereof is selected from the group consisting of immunoglobulin (Ig)A, IgD, IgE, IgG, or IgM.

Example 73. The isolated antibody or active fragment thereof of any of Examples 1-62, further comprising a tag domain fused to the variable domain.

Example 74. The isolated antibody or active fragment thereof of Example 73, wherein the tag domain comprises a fluorescent protein.

Example 75. The isolated antibody or active fragment thereof of Example 73, wherein the tag domain comprises a radiolabel.

Example 76. The isolated antibody or active fragment thereof of Example 73, wherein the tag domain comprises biotin or streptavidin.

Example 77. The isolated antibody or active fragment thereof of any of Examples 1-62, further comprising a chemotherapeutic agent fused to the variable domain.

Example 78. The isolated antibody or active fragment thereof of Example 77, wherein the chemotherapeutic agent is an OPN-c inhibitor.

Example 79. The isolated antibody or active fragment thereof of Example 78, wherein the OPN-c inhibitor is selected from the group consisting of brefelamide, mesalazine, agelastatin A, trastuzumab, methotrexate or combinations thereof.

Example 80. The isolated antibody or active fragment thereof of Example 78, wherein the chemotherapeutic agent is a platinum containing agent.

Example 81. An isolated polynucleotide encoding the isolated antibody or active fragment thereof of any of Examples 1-62.

Example 82. The isolated polynucleotide of Example 81, wherein the polynucleotide comprises a sequence with at least 85% identity to the sequence set forth in SEQ ID NO: 29 or SEQ ID NO: 30.

Example 83. The isolated polynucleotide of Example 81, wherein the polynucleotide comprises a sequence with at least 85% identity to the sequence set forth in in SEQ ID NO: 31 or SEQ ID NO: 32 or SEQ ID NO: 33.

Example 84. The isolated polynucleotide of Example 81, wherein the polynucleotide comprises a sequence with at least 85% identity to the sequence set forth in in SEQ ID NO: 34 or SEQ ID NO: 35 or SEQ ID NO: 36

Example 85. The isolated polynucleotide of Example 81, wherein the polynucleotide comprises a sequence with at least 85% identity to the sequence set forth in SEQ ID NO: 37 or SEQ ID NO: 38 or SEQ ID NO: 39.

Example 86. The isolated polynucleotide of Example 81, wherein the polynucleotide comprises a sequence with at least 85% identity to the sequence set forth in SEQ ID NO: 40 or SEQ ID NO: 41.

Example 87. The isolated polynucleotide of Example 81, wherein the polynucleotide comprises a sequence with at least 85% identity to the sequence set forth in SEQ ID NO: 44 or SEQ ID NO: 45 or SEQ ID NO: 46.

Example 88. The isolated polynucleotide of Example 81, wherein the polynucleotide comprises a sequence with at least 85% identity to the sequence set forth in SEQ ID NO: 47 or SEQ ID NO: 48 or SEQ ID NO: 49.

Example 89. A method of determining OPN-c expression in a tissue, comprising obtaining a tissue sample from a subject and contacting the isolated antibody or active fragment thereof of any of Examples 1-62 to the sample, wherein binding of the composition to the tissue sample confirms expression of OPN-c.

Example 90. The method of Example 89, wherein the sample is of a bodily tissue or a bodily fluid.

Example 91. The method of Example 89 or 90, wherein the sample is a blood sample.

Example 92. The method of any of Examples 89, 90, or 91, wherein the sample is a fractionated blood sample of serum or plasma.

Example 93. A method for determining risk of for malignancy from a pre-malignant breast lesion comprising obtaining a breast tissue sample from a subject with a pre-malignant lesion and contacting the isolated antibody or active fragment thereof of any of Examples 1-62 thereto, wherein binding of the anti-OPN-c composition to the breast tissue sample signals an elevated risk for the subject.

Example 94. The method of Example 93, wherein binding of the isolated antibody or active fragment thereof is by immunohistochemical staining.

Example 95. A method of neutralizing OPN-c expression in a cell or extracellular matrix comprising administering the isolated antibody or active fragment thereof of any of Examples 1-62 to a subject, wherein the isolated antibody or active fragment thereof selectively binds to expressed OPN-c in the subject and neutralizes OPN-c activity within the cell or the extracellular matrix.

Example 96. The method of Example 95, wherein the isolated antibody or active fragment thereof binds an active domain within OPN-c to thereby neutralize function.

Example 97. The method of Example 95, wherein the isolated antibody or active fragment thereof comprises a humanized antibody or fragment thereof.

Example 98. The method of Example 95, wherein the isolated antibody or active fragment thereof comprises a human immunoglobulin framework domain grafted with the CDR sequences as set forth in SEQ ID NO: 1, SEQ ID NO: 19 and SEQ ID NO: 3.

Example 99. The method of Example 95, wherein the isolated antibody or active fragment thereof comprises a human immunoglobulin framework domain grafted with the CDR sequences as set forth in SEQ ID NO: 10, SEQ ID NO: 20 and SEQ ID NO: 12.

Example 100. The method of Example 95, wherein the isolated antibody or active fragment thereof comprises a human immunoglobulin framework domain grafted with the CDR sequences as set forth in SEQ ID NO: 21, SEQ ID NO: 6 and SEQ ID NO: 7.

Example 101. The method of Example 95, wherein the isolated antibody or active fragment thereof comprises a human immunoglobulin framework domain grafted with the CDR sequences as set forth in SEQ ID NO: 22, SEQ ID NO: 16 and SEQ ID NO: 7.

Example 102. The method of Example 95, wherein the isolated antibody or active fragment thereof is fused to a chemotherapeutic agent, whereby the isolated antibody or active fragment thereof selectively binds expressed OPN-c to site specific deliver the chemotherapeutic agent.

Example 103. The isolated antibody or active fragment thereof of any of Examples 1-62, wherein the isolated antibody or active fragment thereof is comprised of an isolated heavy chain (HC) variable domain polypeptide and an isolated light chain (LC) variable domain, wherein the HC variable domain is comprised of three HC complementarity determining regions (CDRs), the first HC CDR (HC CDR1) is comprised of an amino acid sequence as set forth in SEQ ID NO: 1 or an active mutant thereof, the second HC CDR (HC CDR2) being comprised of an amino acid sequence as set forth in SEQ ID NO: 19 or an active mutant thereof and the third HC CDR (HC CDR3) being comprised of an amino acid sequence as set forth in SEQ ID NO: 3 or an active mutant thereof, and further wherein the LC variable domain polypeptide is comprised of three LC complementarity determining regions (CDRs), the first LC CDR (LC CDR1) being comprised of an amino acid sequence as set forth in SEQ ID NO: 21 or an active mutant thereof, the second LC CDR (LC CDR2) being comprised of an amino acid sequence as set forth in SEQ ID NO: 6 or an active mutant thereof, and the third LC CDR (LC CDR3) being comprised of an amino acid sequence as set forth in SEQ ID NO: 7 or an active mutant thereof.

Example 104. The antibody of Example 103, wherein the antibody is a monoclonal antibody.

Example 105. The antibody of Example 103, wherein HC CDR2 comprises an amino acid sequence as set forth in SEQ ID NO: 20.

Example 106. The antibody of Example 103, wherein LC CDR1 comprises an amino acid sequence as set forth in SEQ ID NO: 22.

Example 107. An anti-osteopontin-c (OPN-c) binding composition comprised of an isolated heavy chain (HC) variable domain polypeptide and an isolated light chain (HC) variable domain polypeptide, wherein the HC variable domain is comprised of three HC complementarity determining regions (CDRs), wherein the first HC CDR (HC CDR1) comprises an amino acid sequence as set forth in SEQ ID NO: 1, the second HC CDR (HC CDR2) comprises an amino acid sequence as set forth in SEQ ID NO: 19 and the third HC CDR (HC CDR3) comprises an amino acid sequence as set forth in SEQ ID NO: 3, and further wherein the LC variable domain polypeptide is comprised of three LC complementarity determining regions (CDRs), wherein the first LC CDR (LC CDR1) comprises an amino acid sequence as set forth in SEQ ID NO: 21, the second LC CDR (LC CDR2) comprises an amino acid sequence as forth in SEQ ID NO: 6, and the third LC CDR (LC CDR3) comprises an amino acid sequence as set forth in SEQ ID NO: 7.

Example 108. The anti-OPN-c composition of Example 107, wherein the HC variable domain comprises an amino acid sequence selected from the group consisting of SEQ ID NO: 23, SEQ ID NO: 24 or SEQ ID NO: 25 and the LC variable domain comprises an amino acid sequence selected from the group consisting of SEQ ID NO: 26, SEQ ID NO: 27 or SEQ ID NO: 28.

Example 109. The anti-OPN-c composition of Example 107 or 108, wherein HC CDR2 comprises an amino acid sequence as set forth in SEQ ID NO: 20.

Example 110. The anti-OPN-c composition of Example 107 or 108, wherein LC CDR1 comprises an amino acid sequence as set forth in SEQ ID NO: 22.

The foregoing description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular aspects of the invention, but is not meant to be a limitation upon the practice thereof. The following claims, including all equivalents thereof, are intended to define the scope of the invention.

1. An isolated antibody to osteopontin-c (OPN-c) or an active fragment thereof, wherein the isolated antibody or active fragment thereof comprises an isolated heavy chain (HC) variable domain polypeptide or comprises an isolated light chain (HC) variable domain polypeptide or comprises an isolated HC variable domain and an isolated LC variable domain,
wherein the isolated antibody or active fragment thereof binds a polypeptide with an amino acid sequence as set forth in SEO ID NO: 50; or
a polynucleotide encoding the isolated antibody or active fragment thereof.

2-5. (canceled)

6. The isolated antibody or active fragment thereof of claim 1 comprising the isolated HC variable domain polypeptide, wherein a first HC CDR (HC CDR1) is comprised of an amino acid sequence as set forth in SEQ ID NO: 1, 10, or an active mutant thereof.

7. (canceled)

8. The isolated antibody or active fragment thereof of claim 1 comprising the isolated HC variable domain polypeptide, wherein a second HC CDR (HC CDR2) is comprised of an amino acid sequence as set forth in IGSX₀GSA (SEQ ID NO: 19) or an active mutant thereof, wherein X₀ is any amino acid, or wherein X₀ is an arginine or a serine; or
wherein the second HC CDR (HC CDR2) is comprised of an amino acid sequence as set forth in X₁IGSX₂GSAX₃ASWAKS (SEO ID NO: 20) or an active mutant thereof, wherein X₁ or X₂ or X₃ are any amino acid, or wherein X₁ is glycine or serine, X₂ is arginine or serine, and X₃ is cysteine or tyrosine.

9-13. (canceled)

14. The isolated antibody or active fragment thereof of claim 1 comprising the isolated HC variable domain polypeptide, wherein a third HC CDR (HC CDR3) is comprised of an amino acid sequence as set forth in SEQ ID NO: 3, 12, or an active mutant thereof.

15-16. (canceled)

17. The isolated antibody or active fragment thereof of claim 1, wherein the isolated HC variable domain comprises an amino acid sequence with at least 85% identity to a sequence selected from the group consisting of SEQ ID NO: 23, SEQ ID NO: 24 or SEQ ID NO: 25.

18. (canceled)

19. The isolated antibody to osteopontin-c (OPN-c) for claim 1, wherein the isolated antibody or active fragment thereof comprises an isolated light chain (LC) variable domain polypeptide comprising three LC complementarity determining regions (CDRs);
wherein the first LC CDR (LC CDR1) is comprised of an amino acid sequence as set forth in QX₄VYX₅NW (SEQ ID NO: 21) or an active mutant thereof,
wherein X₄ and X₅ are any amino acid, or
wherein X₄ is a serine or an asparagine, or
wherein X₅ is a lysine or a serine; or
wherein the first LC CDR (LC CDR1) is comprised of an amino acid sequence as set forth in X₄SQX₅VYX₆NWLX₇ (SEO ID NO: 22) or an active mutant thereof, wherein
X₄ or X₅ or X₆ or X₇ can be any amino acid, or
X₄ is serine or asparagine and X₅ is lysine, or serine and X₆ is alanine or serine, and X₇ is valine or alanine.

20-27. (canceled)

28. The isolated antibody or active fragment thereof of claim 1 comprising the isolated light chain (HC) variable domain polypeptide, wherein a second LC CDR (LC CDR2) is comprised of an amino acid sequence as set forth in SEQ ID NO: 6, 16, or an active mutant thereof.

29. (canceled)

30. The isolated antibody or active fragment thereof of claim 1 comprising the isolated light chain (HC) variable domain polypeptide, wherein a third LC CDR (LC CDR3) is comprised of an amino acid sequence as set forth in SEQ ID NO: 7 or an active mutant thereof.

31. (canceled)

32. The isolated antibody or active fragment thereof of claim 1 comprising the isolated light chain (HC) variable domain polypeptide, wherein the isolated LC variable domain comprises an amino acid sequence with at least 85% identity to a sequence selected from the group consisting of SEQ ID NO: 26, SEQ ID NO: 27 or SEQ ID NO: 28.

33-62. (canceled)

63. The isolated antibody or active fragment of claim 1, wherein the isolated antibody or active fragment thereof is a monoclonal antibody.

64. The isolated antibody or active fragment thereof of claim 1, wherein the isolated antibody or active fragment thereof is an Fv fragment, or wherein the isolated antibody or active fragment thereof is an Fab′ fragment.

65-67. (canceled)

68. The isolated antibody or active fragment thereof of claim 1, wherein the isolated antibody or active fragment thereof further comprises a human immunoglobulin framework domain grafted with the CDR sequences as set forth in SEQ ID NO: 1, SEQ ID NO: 19 and SEQ ID NO: 3, or
wherein the isolated antibody or active fragment thereof further comprises a human immunoglobulin framework domain grafted with the CDR sequences as set forth in SEO ID NO: 10, SEO ID NO: 20 and SEO ID NO: 12, or
wherein the isolated antibody or active fragment thereof further comprises a human immunoglobulin framework domain grafted with the CDR sequences as set forth in SEO ID NO: 21, SEO ID NO: 6 and SEO ID NO: 7, or
wherein the isolated antibody or active fragment thereof further comprises a human immunoglobulin framework domain grafted with the CDR sequences as set forth in SEO ID NO: 22, SEO ID NO: 16 and SEO ID NO: 7, or
any combination thereof.

69-71. (canceled)

72. The isolated antibody or active fragment thereof of claim 1, wherein the isolated antibody or active fragment thereof is selected from the group consisting of immunoglobulin (Ig)A, IgD, IgE, IgG, or IgM.

73. The isolated antibody or active fragment thereof of claim 1, further comprising a tag domain or a chemotherapeutic agent fused to the variable domain.

74. The isolated antibody or active fragment thereof of claim 73, wherein the tag domain comprises a fluorescent protein, a radiolabel, biotin, or streptavidin.

75-77. (canceled)

78. The isolated antibody or active fragment thereof of claim 77, wherein the chemotherapeutic agent is an OPN-c inhibitor, or a platinum containing agent.

79. The isolated antibody or active fragment thereof of claim 78, wherein the OPN-c inhibitor is selected from the group consisting of brefelamide, mesalazine, agelastatin A, trastuzumab, methotrexate or combinations thereof.

80-89. (canceled)

90. A method for determining risk of for malignancy from a pre-malignant breast lesion comprising obtaining a breast tissue sample from a subject with a pre-malignant lesion and contacting the isolated antibody or active fragment thereof of claim 1 thereto, wherein binding of the anti-OPN-c composition to the breast tissue sample signals an elevated risk for the subject.

91. (canceled)

92. A method of neutralizing OPN-c expression in a cell- or extracellular matrix comprising administering the isolated antibody or active fragment thereof of claim 1 to a subject, wherein the isolated antibody or active fragment thereof selectively binds to expressed OPN-c in the subject and neutralizes OPN-c activity within the cell or the extracellular matrix.

93-103. (canceled)

104. The anti-osteopontin-c (OPN-c) binding composition of claim 1 comprised of an isolated heavy chain (HC) variable domain polypeptide and an isolated light chain (HC) variable domain polypeptide, wherein the HC variable domain is comprised of three HC complementarity determining regions (CDRs), wherein a first HC CDR (HC CDR1) comprises an amino acid sequence as set forth in SEQ ID NO: 1 or 10, a second HC CDR (HC CDR2) comprises an amino acid sequence as set forth in SEQ ID NO: 19 or 20, and a third HC CDR (HC CDR3) comprises an amino acid sequence as set forth in SEQ ID NO: 3 or 12, and further wherein the LC variable domain polypeptide is comprised of three LC complementarity determining regions (CDRs), wherein a first LC CDR (LC CDR1) comprises an amino acid sequence as set forth in SEQ ID NO: 21 or 22, a second LC CDR (LC CDR2) comprises an amino acid sequence as forth in SEQ ID NO: 6 or 16, and a third LC CDR (LC CDR3) comprises an amino acid sequence as set forth in SEQ ID NO: 7.

105-107. (canceled)
